(19) United States
(12) Patent Application Publication
(10) Pub. No.: US 2017/0082625 A1
(43) Pub. Date: Mar. 23, 2017

(54) METHOD AND SYSTEM FOR MICROFILTER-BASED CAPTURE AND RELEASE OF CANCER ASSOCIATED CELLS

(71) Applicant: UNIVERSITY OF MIAMI, Miami, FL (US)

(72) Inventors: Ashutosh Agarwal, Miami Beach, FL (US); Zheng Ao, Miami, FL (US); Richard Cote, Miami, FL (US); Ram H. Datar, Miami, FL (US)

(21) Appl. No.: 15/269,574

(22) Filed: Sep. 19, 2016

Related U.S. Application Data
(60) Provisional application No. 62/219,808, filed on Sep. 17, 2015.

(51) Int. Cl. G01N 33/574; G01N 1/44; G01N 1/40

(52) U.S. Cl. CPC G01N 33/574; G01N 1/405; G01N 1/4077; G01N 1/44; G01N 2001/4088

(57) ABSTRACT

A slotted microfilter is coated with a phase changeable material having a hydrophobic state under a first temperature and a hydrophilic state under a second temperature. An example coating material is poly(N-isopropylacrylamide) (“PIPAAm”). The microfilter can be controlled to switch between a capture state where circulating tumor cells are captured by the microfilter and a release state, where viable tumor cells are released from capture for analysis, e.g., single cell phenotypic and genomic analysis, or for ex vivo culture growth.

### CROSS-REFERENCE TO RELATED APPLICATION

This application claims the benefit of U.S. Provisional Application Ser. No. 62/219,808, filed Sep. 17, 2015, entitled “Method and System for Microfilter-Based Capture and Release of Cancer Associated Cells,” which is hereby incorporated by reference in its entirety.

### STATEMENT OF GOVERNMENT SUPPORT

This invention was made with government support under 5R21CA182050-02 awarded by the National Institutes of Health. The Government has certain rights in the invention.

### FIELD OF THE INVENTION

The present disclosure relates to capturing cells and, more particularly, to using a coated microfilter functionalized to controllably capture cells and release captured cells.

### BACKGROUND

The background descriptions provided throughout are for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

The Circulating Tumor Cell (CTC) is an important biomarker in cancer management. Enumeration of CTCs has been proven of prognostic value in multiple cancer types including breast, prostate and colorectal cancer. Recent studies, however, have revealed the limited clinical relevance of CTC enumeration alone in interventional trials. Additionally, single CTC transcriptomic studies have revealed the wide heterogeneity of CTCs, further indicating the need for in depth molecular and functional characterization of CTCs. Indeed, there have been increased efforts to implement molecular and genomic characterization of CTCs for informing clinical trial design, treatment selection, and ultimately, precision cancer management. To fulfill such goals, a technology that not only allows for efficient CTC capture but also versatility to accommodate various downstream characterization of CTC both on-chip and off-chip is highly desirable.

### SUMMARY OF THE INVENTION

In accordance with an example, a system for capturing and releasing cells from a sample, the system comprises: a microfilter device comprising a membrane filter having a substrate and an array of filtering holes each of a predetermined shape and dimension, the filtering holes forming an array pattern in the substrate, the substrate having a first surface and an opposing second surface, the first surface being positioned to provide a cell capture surface; a poly(N-isopropylacrylamide) coating (“PIPAAm coating”) deposited on the first surface and functionalized (i) to remain attractive to cells while cells are captured based on size through a non-specific electrostatic binding, during a hydrophilic state in which the PIPAAm coating is at a temperature at or below a capture temperature and (ii) to release the cells through a phase transition, during a hydrophobic state in which the PIPAAm coating is at a temperature at or above a release temperature, where the release temperature is greater than the capture temperature.

In accordance with another example, a method for capturing and releasing cells from a sample, the method comprises: providing the sample to a microfilter device comprising a membrane filter having a substrate and an array of filtering holes each of a predetermined shape and dimension, the filtering holes forming an array pattern in the substrate, the substrate having a first surface and an opposing second surface, the first surface being positioned to provide a cell capture surface, wherein the member is coated with a poly(N-isopropylacrylamide) coating (“PIPAAm coating”) deposited on the first surface; with the membrane at or below a first temperature corresponding to a hydrophilic state, capturing cells in the membrane through a non-specific electrostatic binding; selectively heating the membrane to or above a second temperature corresponding to a hydrophobic state and releasing the captured cells during the hydrophilic state, wherein the second temperature is equal to or greater than the first temperature; and collecting the released captured cells for analysis.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1A illustrates a top, microscopic view (400× magnification) of a (PIPAAm) coated slotted filter, in accordance with an example.

FIG. 1B illustrates a filtration system using a pumping mechanism, e.g., a syringe pump, to capture CTCs from blood using the coated slotted filter of FIG. 1A.

FIG. 1C illustrates a coated slotted filter assembly with a (PIPAAm coated) slotted microfilter sandwiched in a cassette housing, in accordance with example.

FIG. 1D illustrates a process for using temperature change to release captured CTCs from a coated slotted filter.

FIG. 2A illustrates fluorescence microscopy taken before release of CTCs and after release of CTCs from, in particular before and after the release of SK-Br-3 breast cancer cells captured and released from blood using a PIPAAm coated slotted filter. The SKBr-3 cells released from the coated slotted filter were tested with a Live-Dead Assay showing that 95% (540 out of 567 cells counted) of the cells remained viable (Green) following release from the filter. The dead cells are labeled in red.

FIG. 2B illustrates phase contrast microscopy images taken of released CTCs expanded in culture for 3 days and 10 days after release, showing the viability of the CTC cells.

FIG. 3A illustrates the capture of lung carcinoma that had metastasized to the spine stably labeled with green fluorescent protein (LMTS-GFP) CRCs using a coated slotted filter, with the cells cultured on a Parylene C surface. Parylene C surfaces do not support growth of LMTS-GFP CRCs.

FIG. 3B illustrates the capture of LMTS-GFP CRCs using a coated slotted filter, with the cells cultured on a culture plate.

FIGS. 3C and 3D are phase contrast microscopy images taken at 1 day (FIG. 3C) and 5 days (FIG. 3D) of LMTS-GFP cells captured and released by a coated slotted filter.

FIGS. 4A and 4B are fluorescence microscopy images showing proliferation rates and metabolism rates of tumor cells before and after capture and release from a coated slotted filter. Proliferation rate of the LMTS-GFP cells was measured using an EdU assay. All cells were labeled with Hoechst (Blue) and express GFP (Green), and newly proliferated cells were labeled with EdU (Red). FIG. 4A illustrates a control using LMTS-GFP cells plated conventionally without passing through a filter. FIG. 4B illustrates a spike in LMTS-GFP cells retrieved when using a coated slotted filter (specifically a PIPAAm coated filter).

FIGS. 4C and 4D illustrate growth curves of plots SKBr-3 cells (FIG. 4C) and LMTS-GFP cells (FIG. 4D) measured using an MTT Assay. No significant differences in metabolism rate and proliferation rate were seen between released cells from the (PIPAAm) coated slotted filter and control cells for either SKBr-3 cells (FIG. 4C) or the LMTS-GFP CRC line (FIG. 4D).

FIG. 4E illustrates the captured and released LMTS-GFP cells for mutated p53 protein shown in control cells. Lane 1 shows extracts from a parental control. Lane 2 shows LMTS-GFP cells cultured following release from a (PIPAAm) coated slotted filter and probed by immunoblot for the p53 tumor suppressor protein. This LMTS line harbors an E298* truncation mutation with a predicted molecular weight of approximately 43 kDa (asterisk). Lane 3 shows LNCaP cells run as a control for wild type p53 (arrow). β-actin was probed as loading control as seen on bottom panel FIG. 4E.

FIG. 5 is a table showing capture, release and retrieval efficiency of a PIPAAm coated slotted filters. Capture efficiency was calculated by dividing cell numbers captured on filter before release by cell numbers spiked into blood. Release efficiency was calculated by dividing cell numbers released from the filter by cell numbers captured on the filter before release. Retrieval Efficiency was calculated by dividing cell numbers released from filter by cell numbers spiked into blood.

FIG. 6A is a phase contrast microscopy image of a slotted filter before coating, and FIG. 6B is phase contrast microscopy image of a slotted filter after coating with PIPAAm. Pore lengths for each and the differences are shown in the table of FIG. 6C.

FIGS. 7A and 7B illustrate phase contrast microscopy images. Contaminating erythrocytes and leukocytes were removed by gentle washes. Approximately 1000 SKBr-3 cells were retrieved from blood by using a PIPAAm coated slotted filter and the cells were plated on a 48-well plate. FIG. 7A shows the culture at 16 hours, with SKBr-3 tumor cells adhered to culture plate (green arrows), whereas apoptotic erythrocytes and leukocytes were also settling at the bottom of the plate (red arrows). FIG. 7B illustrates a post-wash, non-adherent cells were removed, leaving adherent tumor cells on the plate (green arrows).

FIG. 8 illustrates a process for circulating tumor cell capture and release using a PIPAAm coated slotted filter.

FIG. 9 are images showing the proliferation rates of SKBr-3 cells compared before and after PIPAAm filter capture and release, as measured using an EdU assay. FIG. 9A illustrates SKBr-3 cells plated as controls, without filtering. FIG. 9B illustrates SKBr-3 cells retrieved from blood using a PIPAAm coated slotted filter.

FIG. 10 is a table showing a calculation of CTC capture and release rates using a pre-labeled SK-Br-3 cells spiked in healthy donor's blood as a model system. Three replicates were performed on different dates and calculations were done within each set of replicates.

FIG. 11 is a table showing calculation of capture and release rates with LMTS-GFP cells spiked in a healthy donor's blood as a model system. Three replicates were performed on the same date with the same aliquots, so calculations were done on averaged numbers with each replicate.

FIG. 12 is a table showing calculation of SKBr-3 capture and release rates using non-coated filters with SKBr-3 cells spiked in a healthy donor's blood as a model system. Three replicates were performed.

FIG. 13A illustrates enumeration of CTCs from metastatic breast cancer patients captured using round-pore filters or PIPAAm coated slotted filters. Samples were subjected to immunefluourescence imaging for markers of Pan-cytokeratin (Alexa 488-Green) and CD45 (Alexa 680-White). The sample was then counter-stained with DAPI (Blue). CTCs were identified as DAPI+CK+CD45− cells.

FIG. 13B illustrates CTC enumeration and release data using round-pore filters and PIPAAm coated slot pore filters.

### DESCRIPTION

The present techniques describe a cell collecting apparatus based on a capturing microfilter. The microfilter uses a temperature responsive polymer coating to achieve both capturing (and culturing) of cells and release of cells, including viable CTCs captured from patient blood samples. In particular, we have discovered a way of using temperature responsive polymers in an antigen-agnostic filtration based platform through the use of a poly(N-isopropylacrylamide) (“PIPAAm”) coating, capable of both capture and release of viable CTCs from blood at high efficiencies. The techniques, therefore, provide the foundation for in-depth characterization of viable CTCs, including single cell phenotypic and genomic analysis as well as ex vivo CTCs culture growth.

The present techniques stand in stark contrast to conventional systems for cell capture. For example, several platforms that allow for viable CTC capture and release have been reported based on immobilized antibodies conjugated to a cleavable linker, Poly (N-iso-propylacrylamide) (PIPAAm), or electroactive films. However, these systems are affinity-based and can be potentially biased by the target antigen. For example, the most commonly targeted antigen for CTC capture, EpCAM, can be absent within certain CTC populations possessing mesenchymal phenotypes.

In contrast to these conventional systems, the present techniques provide a label free, size-based isolation and release of CTCs that allow for the study of CTC populations in a more comprehensive manner. The PIPAAm coated filtration-based platforms described herein are able to undergo a state change process, from a hydrophilic state (or capturing state) where cells (e.g., CTCs) are captured in a microfilter, to a hydrophobic state (or release state) where the captured cells are controllably released.

PIPAAm is a temperature responsive polymer that undergoes a reversible lower critical solution temperature (LCST) phase transition at a solution temperature of 32° C. Traditionally, this property of PIPAAm has been used for tissue engineering applications.

Typically, cells are cultured on PIPAAm coated surfaces at 37° C. when PIPAAm is hydrophobic. The cells can then be detached as a sheet when the culture temperature is shifted to below 32° C., where the PIPAAm coated surface becomes hydrated.

We have developed a cell capture apparatus that uses PIPAAm in a contra-indicated way, compared to conventional uses. With the present techniques, tumor cells, e.g., epithelial cancer cells, are bound to the parylene C membrane by non-specific electrostatic interactions, which is a quite different mechanism that extracellular matrix (ECM) mediated adhesion. The use of an entirely different cell adhesion mechanism means a counter-intuitive PIPAAm-based release strategy was developed. With the present techniques, epithelial cell capture is performed at room temperature (i.e., below 32° C.) in the hydrophilic state, and cell release is enabled by placing the coated filter in a culture media maintained at 37° C. At this temperature, the PIPAAm polymer layer becomes hydrophobic, thereby releasing the electrostatically bound cells. The cells may be captured from cells from any suitable body fluids, including blood, urine, ascites, etc., and may be captured by size.

Applications of the present technology include the capture of patient-derived CTCs from blood and release of them with minimal impact on their viability. While examples herein discuss CTC capture, any type of patient-derived circulating tumor cell may be captured by the microfilter, for culture purposes and molecular characterization purposes or otherwise. Example captured tumor cells include CTCs, circulating cancer associated fibroblasts (cCAFs), CTCs forming clusters with cCAFs, CTCs clustering with leukocytes, and cCAFs alone clusters. Therefore, discussions herein of examples related to CTC capture are intended to include capture of these other cells types more broadly. As such, the methodologies described herein are useful for the isolation, enumeration, and establishment of CTCs, cCAFs, etc. as viable cultures that can be studied and manipulated.

Accordingly, in some aspects, the disclosure provides a method of detecting tumor cells (e.g., CTCs, cCAFs, etc.) in a patient, the method comprising obtaining a sample from the patient; capturing the tumor cells in the sample via a cell-size based microfilter, in a hydrophilic state; and controllably releasing and detecting those captured tumor cells, in a hydrophobic state.

In any embodiment of the disclosure, the sample may be a peripheral blood sample.

In some embodiments, the patient has cancer. In related embodiments, the cancer is breast cancer, colorectal cancer, a sarcoma, a hematopoietic cancer, a neurological malignancy, or prostate cancer.

In some aspects, a method of viably detecting, capturing and releasing of tumor cells, e.g., CTCs or cCAFs, circulating endothelial cells, mesenchymal stem cells, etc. in a patient is provided, the method comprising obtaining a peripheral blood sample from the patient; and detecting the tumor cell and other circulating cells that can be enriched through a size based strategy, in the sample by capturing tumor cells and after a given time period controllably releasing those tumor cells. In some embodiments, the detecting is performed via a cell-size based microfilter.

In further aspects, the disclosure provides a method of identifying metastasis in a patient, the method comprising obtaining a sample from the patient; detecting the tumor cell (e.g., CTCs or cCAFs) in the sample by capturing the tumor cells and after a given time period controllably releasing the tumor cells; and enumerating the tumor cells; wherein the enumerating identifies whether metastasis is present in the patient.

In any of the embodiments of the disclosure, the diameter of captured and released tumor cells (e.g., CTCs or cCAFs) is about 10 microns (μM) or greater. In further embodiments, the size of a tumor cell is from about 10 μm to about 200 μm, or from about 10 μm to about 150 μm, or from about 10 μm to about 100 μm, or from about 10 μm to about 50 μm, or from about 10 μm to about 30 μm in diameter. In still further embodiments, the diameter of a tumor cell is or is at least about 10 μm, is or is at least about 20 μm, is or is at least about 30 μm, is or is at least about 40 μm, is or is at least about 50 μm, is or is at least about 60 μm, is or is at least about 70 μm, is or is at least about 80 μm, is or is at least about 90 μm, or is or is at least about 100 μm in diameter. In some embodiments, the diameter of a tumor cell is less than about 20 μm, less than about 30 μm, less than about 40 μm, less than about 50 μm, less than about 60 μm, less than about 70 μm, less than about 80 μm, less than about 90 μm, or less than about 100 μm in diameter.

The disclosure also contemplates that tumor cells (e.g., CTCs or cCAFs) cluster. Thus, in various embodiments, a cluster of tumor cells contains from about 2 to about 100 cells, or from about 2 to about 80, or from about 2 to about 50, or from about 2 to about 20, or from about 2 to about 10, or from about 2 to about 5 tumor cells. In some embodiments, a cluster of tumor cells contains at least 2, at least 3, at least 4, at least 5, at least 6, at least 7, at least 8, at least 9, at least 10, at least 20, at least 50, at least 70, or at least 80 cells. In specific embodiments, a cluster of tumor cells contains 2, 3, 4, 5, 6, 7, 8, 9, 10, 12, 15, 20, 30, 40, 50, 60, 70, 80, 90, 100 cells or more. The diameter of such clusters of tumor cells is from about 20 μm to about 1000 μm. In further embodiments, the diameter of a cluster of tumor cells is from about 20 μm to about 950 μm, or from about 20 μm to about 900 μm, or from about 20 μm to about 850 μm, or from about 20 μm to about 800 μm, or from about 20 μm to about 750 μm, or from about 20 μm to about 700 μm, or from about 20 μm to about 650 μm, or from about 20 μm to about 600 μm, or from about 20 μm to about 5500 μm, or from about 20 μm to about 500 μm, from about 20 μm to about 450 μm, or from about 20 μm to about 400 μm, or from about 20 μm to about 350 μm, or from about 20 μm to about 300 μm, or from about 20 μm to about 250 μm, or from about 20 μm to about 200 μm, or from about 20 μm to about 150 μm, or from about 20 μm to about 100 μm, or from about 20 μm to about 50 μm. In specific embodiments, the diameter of a cluster of tumor cells is about 20 μm, about 30 μm, about 40 μm, about 50 μm, about 60 μm, about 70 μm, about 80 μm, about 90 μm, about 100 μm, about 150 μm, about 200 μm, about 250 μm, about 300 μm, about 350 μm, about 400 μm, about 450 μm, about 500 μm, about 600 μm, about 700 μm, about 800 μm, about 900 μm, about 950 μm, about 1000 μm or more.

In further aspects, the disclosure provides a method of determining effectiveness of cancer treatment in a patient, the method comprising obtaining a sample from the patient; detecting the tumor cells (e.g., CTCs or cCAF) in the sample by capturing tumor cells and after a given time period controllably releasing the tumor cells; enabling culturing of the tumor cells; and downstream molecular and functional analysis of released tumor cells including, gene expression analysis via q-PCR, single cell microfluidics/isolation platform such as Fluidigm™, DEPArray™, CellCelector™, genomic/transcriptomic sequencing, digital PCR analysis, NanoString™ analysis, mass-spectrum analysis, and drug sensitivity test. Information gained through downstream analysis could be used towards drug screening, novel therapeutics development, patient prognosis, treatment effect monitoring and treatment decision-making.

The microfilter, as described, may be a parylene microfilter, as described, that includes a membrane having an array of holes (e.g., formed as slots) to allow for blood flow at CTC capture. The holes may be of any of a range of shapes, sizes, densities, patterns, and orientations. Example filters are described in U.S. Pat. Nos. 7,846,393, 7,846,743, 8,114,289, and 8,551,425 and PCT Application No. PCT/US06/15501, the entire specifications of each of which are hereby incorporated by reference. The microfilter may be formed of any polycarbonate material, and may be formed of silicone, polyimide, polyurethane, or hydrophilic photoresist such as su-8. In other examples the microfilter may be formed of a metal, such as nickel.

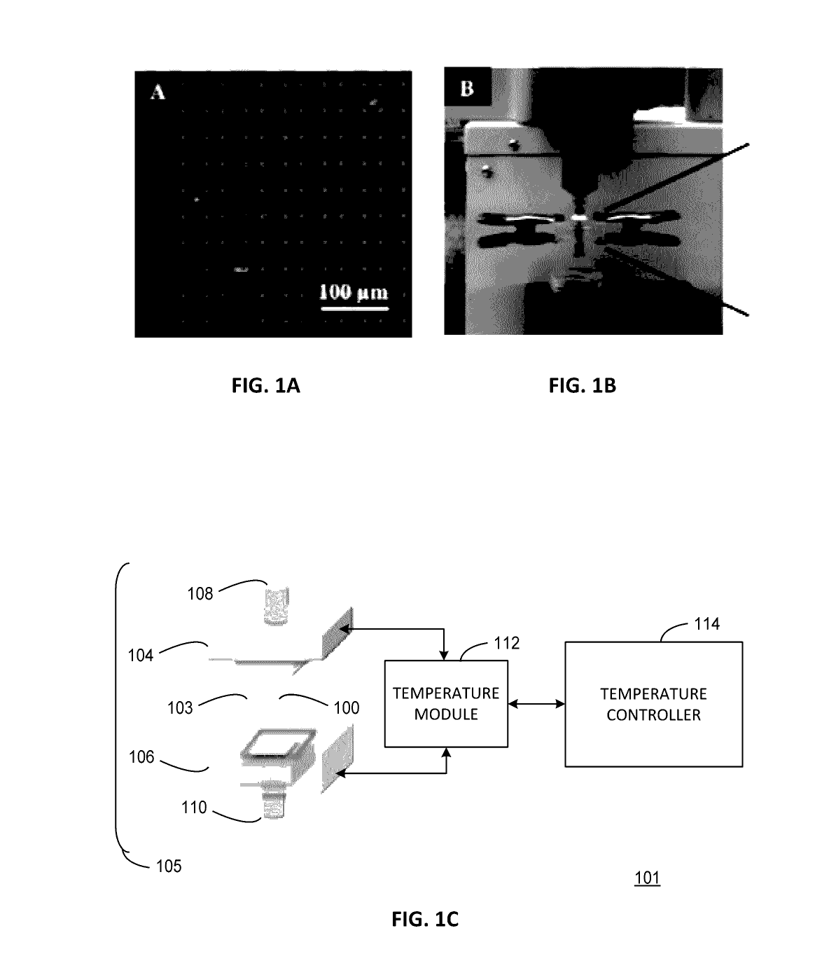

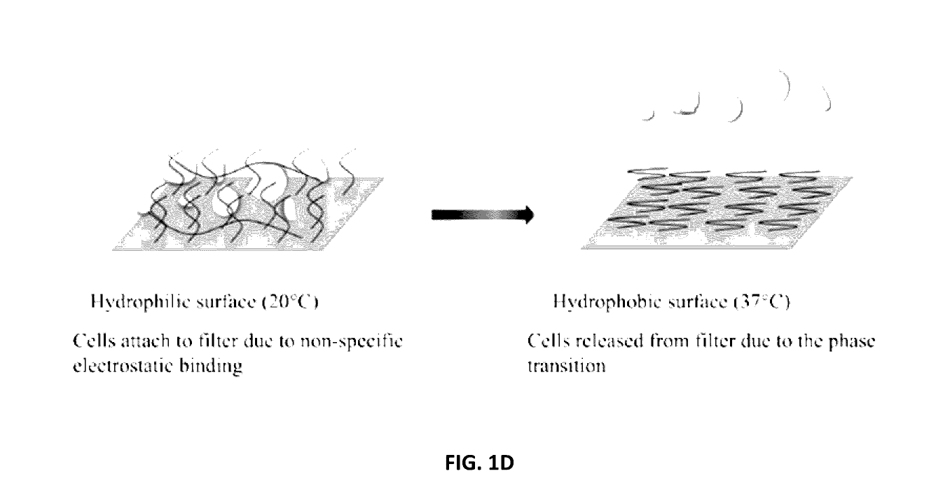

FIG. 1A illustrates a PIPAAm coated slotted microfilter 100 to capture and release circulating tumor cells from blood. FIG. 1A shows a microscopic view (400× magnification) of the slot microfilter 100 after having been coated with PIPAAm. FIG. 1B shows an example filtration set-up with a syringe pump 102 to capture CTCs from blood using the PIPAAm coated slot microfilter 100, with outer cassette frame 103.

FIG. 1C shows a CTC capture device 101 including the PIPAAm coated slot microfilter 100, which is sandwiched between a top cassette 104 of a housing module 105 and a bottom cassette 106 of a housing module 105. Each cassette 104 and 106 includes a tube inlet 108 and an outlet tube 110, respectively, for facilitating blood flow across the microfilter 100, and aligned to maximize flow and CTC capture by the microfilter 100. The blood flows from the tube inlet 108 through the filter 100, acting as a state-adjustable capture stage, before the blood flows enters the outlet tube 110, where it the blood may be recirculated into the patient in vivo or through the ex vivo blood transmission mechanism. Cassette frame 103 is sized and configured to mount within an internal housing chamber positioned inside of one or both of the cassettes 104 and 106. FIG. 1D illustrates a process of using temperature change to release captured CTCs from PIPAAm coated slotted filters. The microfilter 100 has been coated with a material having two, temperature controlled states: a hydrophilic surface state, at a first, lower temperature, where an activation of non-specific electrostatic binding occurs, resulting in filter capture of a target such as CTCs; and a hydrophobic surface state, at a second, higher temperature, in which cells captured in the hydrophilic state are released from the filter due to a phase transition. The microfilter 100 may be coated over a top and a bottom surface, or only on one surface of the two, for example, on the first surface adjacent the inlet tube 108.

While FIG. 1A shows a particular configuration and pattern for the slots forming the slotted microfilter 100, the pattern may vary from filter to filter. Further, the orientation of the slots may vary. Further still, different orientations of slots may be implemented and/or different orientations of the slots may be implemented on a single microfilter.

The device 101 for capturing tumor cells (e.g., CTCs, cCAFs, etc.) includes a controllable temperature module 112, which may be external to or embedded into one or both of the cassettes 104, 106. The temperature module 112 is configured to control the temperature of the microfilter 100 or its PIPAAm coating layer(s) to control the state of the filter between the hydrophobic and hydrophilic states. The module is controlled by a computer processing device 114 (with at least one computer processor and at least one tangible storage medium or memory storing instructions executable by the at least one computer processor). The computer processing device 114 includes instructions to switch the state of the microfilter 100 by adjusting the temperature through instructions sent to the temperature module 112, which can adjust its temperature from a capture temperature below 32° C. to a release temperature above 32° C. In some examples, the temperature module includes not just a temperature source by a temperature sensor for feedback based temperature control.

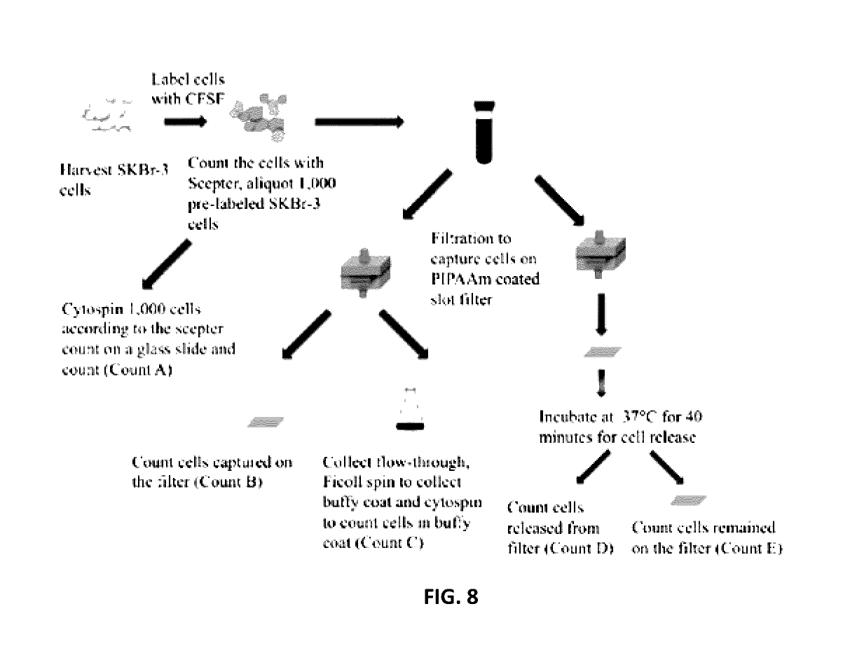

FIG. 8 illustrates an example process for tumor cell capture and release using a PIPAAm coated slotted filter, with an empirical example. First SK-Br-3 breast cancer cells (from ATCC in Manassas, Va.) were labeled with a green fluorescence using CellTrace CFSE Cell Proliferation Kit (Gibco, Life Technologies, Grand Island, N.Y.). Approximately 1000 of the pre-labeled fluorescence Sk-Br-3 cells, based on cell count using Scepter Automated CII Counter (from Millipore of Gibbstwon, N.J.) were spiked into 3 ml of healthy donor's blood. One third of the cell suspension was spun onto a glass slide for cell counting under fluorescence microscopy to quantify and verify the number of cells in the blood (Count A). The remaining spiked blood was then diluted 1:1 with 3 mL of Hank's Balanced Salt Solution (HBSS (Gibco) and two equal aliquots of the cell suspension were processed in parallel through PIPAAm coated slotted filters at a constant flow rate of 75 mL/hour. Post capture, 1 mL of sterile HBSS was filtered through at the same flow rate to remove remaining red blood cells and debris. Next, one filter was directly mounted on a glass coverslip and examined under the fluorescence microscope to enumerate the number of cells captured on filter (Count B). The flow-through of the sample was also examined for cell loss during filtration (Count C). For the second sample, we reversed the filtration cassette and used 1 mL of McCoy's 5A culture medium (Gibco) to flush out cells trapped in the pores at a constant flow rate of 100 mL/hour. Post-reverse flow, the filter was then incubated in culture medium at 37 C (using a VWR symphony incubator of Radnor, PA) for 20 minutes. Post incubation, the release medium was spun onto the glass slide using a Statspin Cytofuge (from Beckman Coulter of Miami, Fla.) for enumeration of the cells released from the filter (Count D). The filter was examined, post cell release, to insure effective cell collection (Count E).

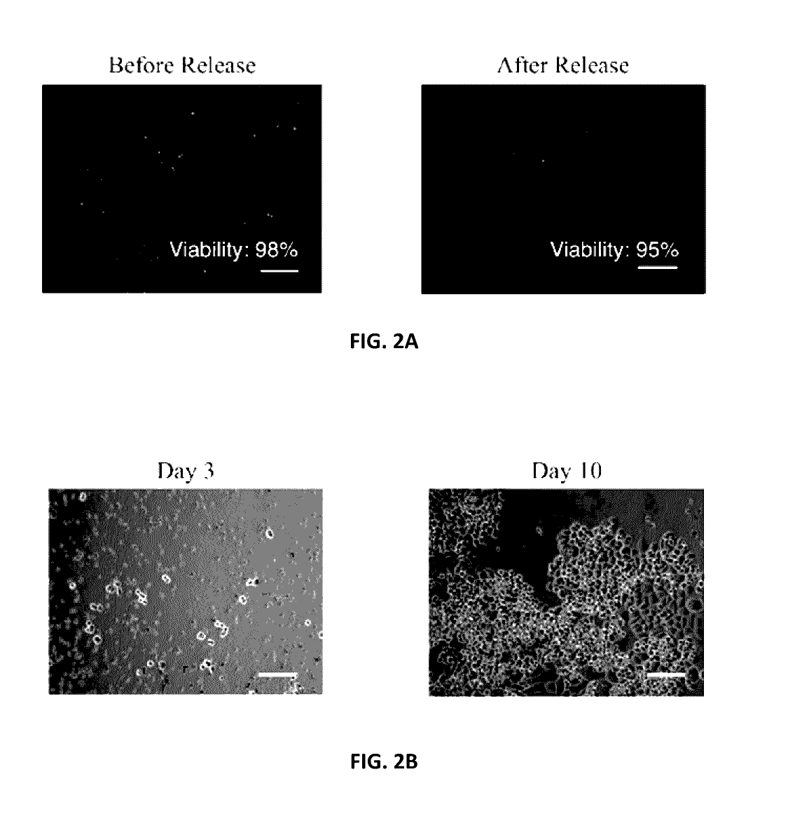

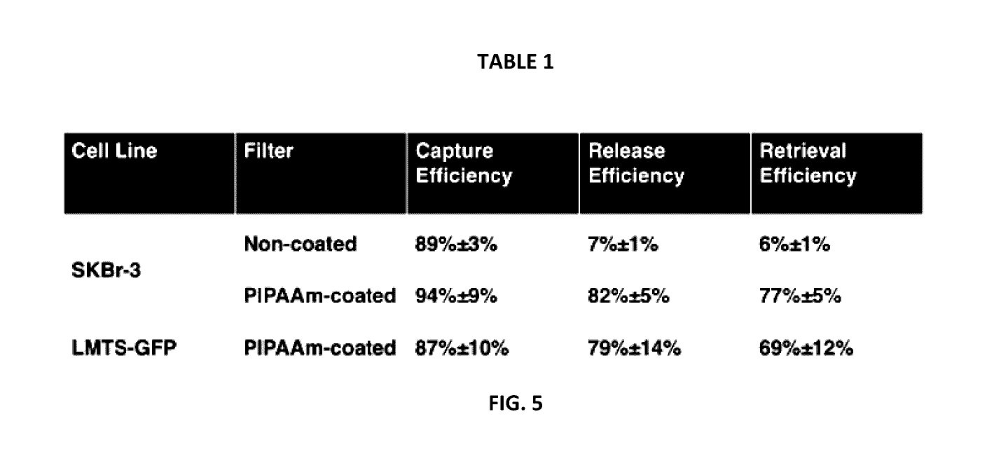

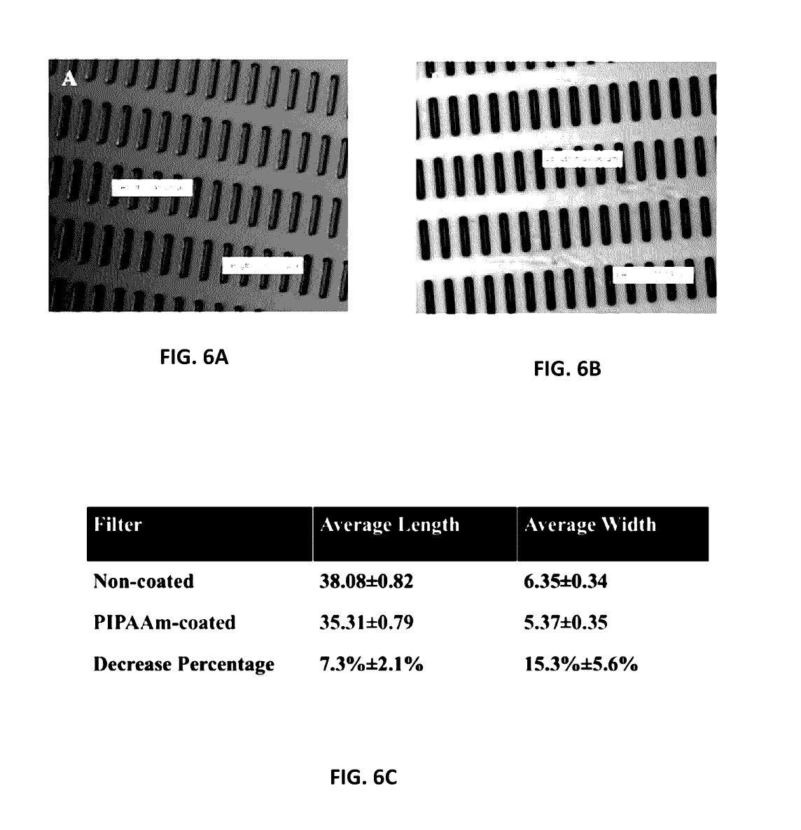

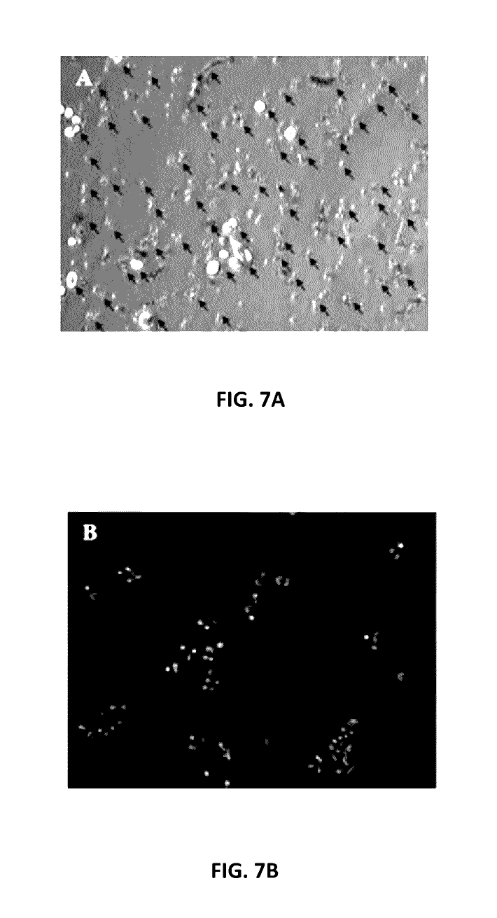

To coat PIPAAm onto the slotted filter 100, in an example, we first dissolved PIPAAm in butanol at 10% (w/v) concentration. The PIPAAm solution was then spin coated onto the slotted filter 100 at a top speed of 6000 rpm for one minute. The coated filter 100 was air-dried and stored at room temperature before use. We compared the pore sizes before and after coating by phase-contrast microscopy FIGS. 6A and 6B (data in FIG. 6C) and as anticipated, found a 7% decrease in pore length and 15% decrease in pore width after PIPAAm coating (FIG. 6c). The effect of this pore size decrease was enhancement in capture efficiency (Table 1, shown in FIG. 5), along with a decrease in enrichment factor, with more erythrocytes and leukocytes seen post capture (FIG. 7A). This is consistent with the test on slotted filters with a 5 μm pore width. However, this decrease in enrichment factor does not hamper the functionality of the PIPAAm coated slotted filter 100 for viable CTC capture and release, since the additional erythrocytes and leukocytes captured can be easily removed by a gentle wash of fresh media at day 1 (FIG. 7B). This did not affect cell viability, proliferation or metabolic rate post capture (FIGS. 2, 4, and 9).

To perform CTC capture in an example, the coated filter was cut into 6 mm by 6 mm squares and fit into a filtration cassette (Top acrylic piece: H 4 mm, L 30mm, W 18 mm, bottom acrylic piece: H 8 mm, L 30 mm, W 18 mm). Methodologically, CTCs are captured onto PIPAAm coated slotted filters at room temperature with PIPAAm coating in its hydrophilic state. Post capture, a mild reverse flow was applied to release cells trapped in the pores and then the filter was incubated in the release medium at 37° C. to induce the phase transition. The cells bound to filter are then released.

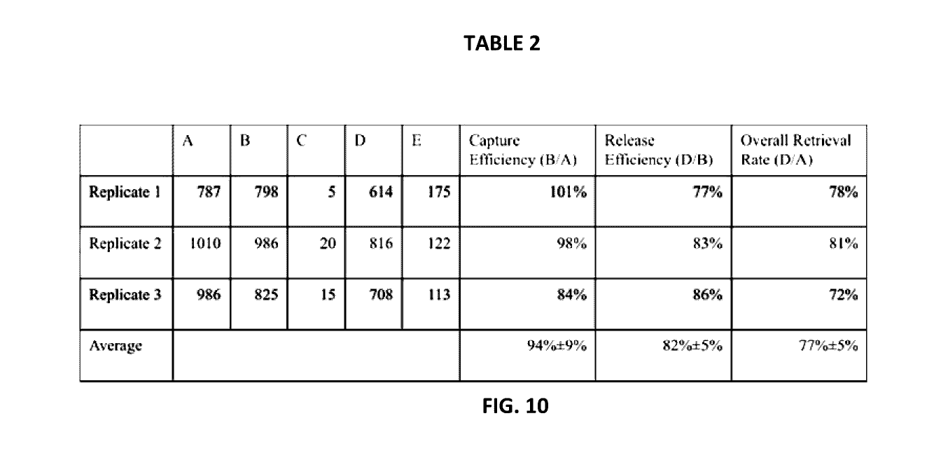

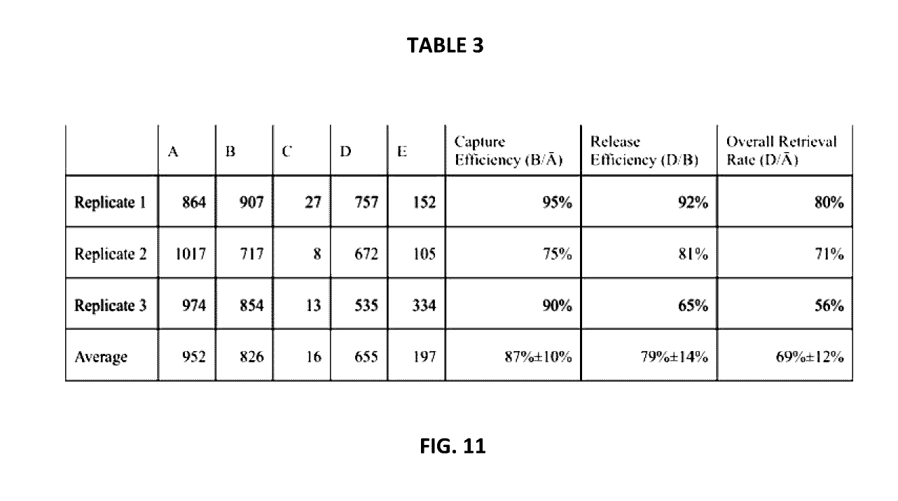

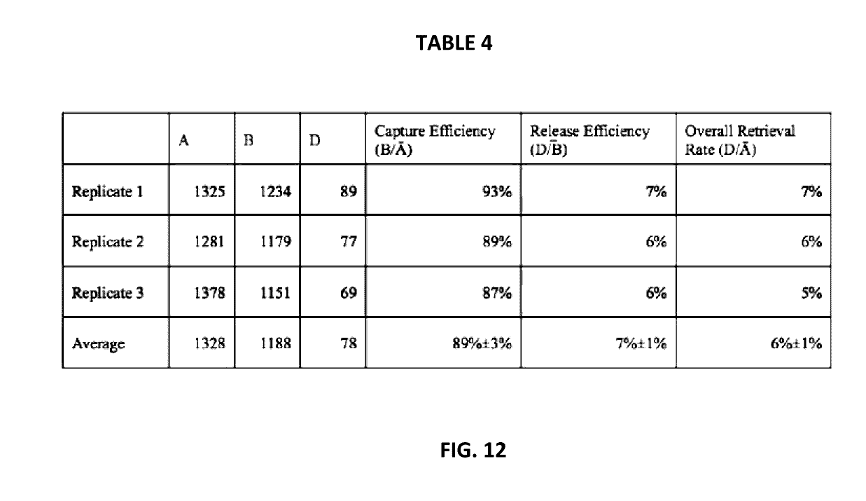

To test the PIPAAm coated slotted filters, we performed detailed analyses of tumor cell capture and release efficiency using ˜1,000 SKBr-3 cells labeled by Carboxyfluorescein Succinimidyl Ester (CFSE) spiked into 7.5 mL of healthy donor's blood as a model system. Results from 3 replicates are shown in FIG. 10, Table 2. The coating method did not hamper the capture efficiency of the filter itself. Overall, we achieved capture, release and retrieval efficiency averages of 94%±9%, 82%±5% and 77%±5% respectively (Table 1, summarized from data in FIG. 10, Table 2 and FIG. 11, Table 3. The release and retrieval efficiency was increased as compared with those of non-coated filter (7%±1% release efficiency and 6%±1% retrieval efficiency) (Table 1, FIG. 12, Table 4).

To test the viability of the cells released from the filter, we spiked ˜1,000 SK-Br-3 cells into 7.5 mL of healthy donor's blood, captured and released them from PIPAAm coated slotted filter using the method described above. A Live-Dead® assay (Life Technologies, Grand Island, N.Y.) was performed to evaluate the cell viability before spike into blood and after release. The pre-spike viability was 98% (592 out of 602 cells counted) and the viability of cells captured and released from blood was 95% (540 out 567 cells counted) (FIG. 2A). We also cultured, in parallel experiment, the cells released from blood McCoy's 5A culture medium (Gibco, Life Technologies, Grand Island, N.Y.). Images were taken at day 3 and day 10. As shown in FIG. 2B, cells released from the filter remained viable and expanded rapidly in culture establishing their viability post cell capture from blood and release from the filter.

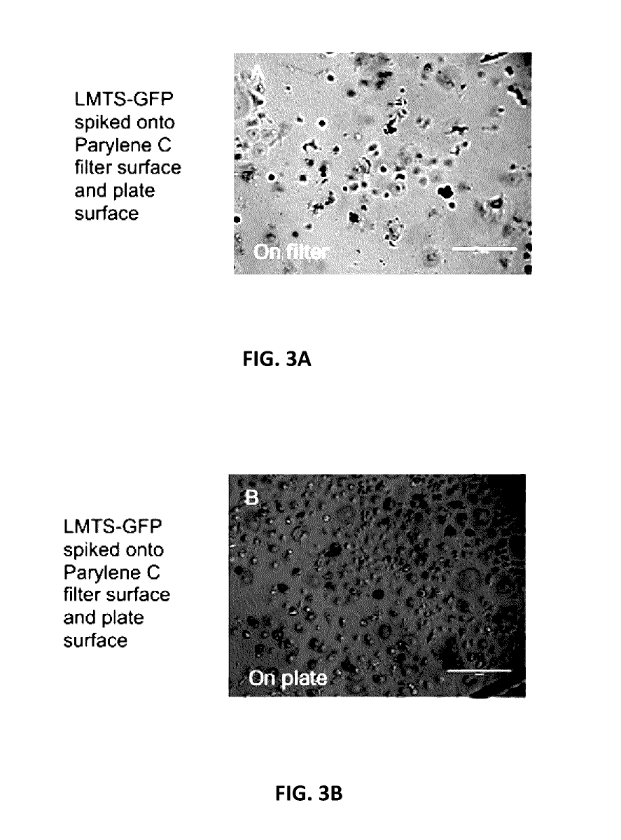

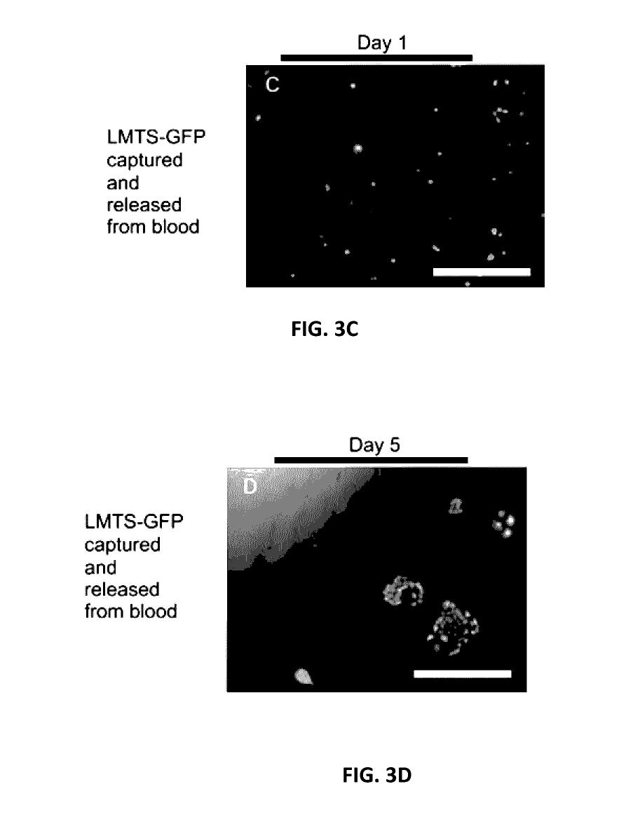

The application of this technology is to capture patient-derived CTCs from blood and release them with minimal impact on their viability. The released CTCs can then be subject to optimal culture surfaces/conditions, which would have been impossible on cells retained on filter. To demonstrate the feasibility of this application, we used conditionally reprogrammed cells (CRCs) as a model system. The CRC approach enables the establishment of continuous primary cell cultures from the majority of the epithelial tissues and could potentially be applied for CTCs culture establishment. Using the CRC method, a tumor cell line was established from a non-small cell Lung carcinoma that had Metastasized To the Spine (LMTS). This line was stably labeled with green fluorescent protein (GFP) and termed LMTS-GFP. Although Parylene C can be favorable for culturing certain immortalized cell line, it was not the optimal surface to culture LMTS-GFP cells (FIG. 3A). Thus, in order to retrieve LMTS-GFP cells from blood and expand them in culture, we tested the PIPAAm coated slotted filter for viable cell release, post capture. First, we tested the capture, release and retrieval efficiency of LMTS-GFP cells on the PIPAAm coated filters. ˜1,000 LMTS-GFP cells were spiked into 7.5 mL of healthy donor's blood and the capture and release experiment was performed in triplicate. As indicated in Table 1 (FIG. 5), efficiencies of capture, release and retrieval produced averages values of 87%±10%, 79%±14% and 69%±12%, respectively. With the confirmative results showing high efficiency retrieval of LMTS-GFP cells from blood, we then tested the culture of these cells retrieved from blood. We spiked ˜1,000 LMTS-GFP cells into 3 mL of healthy donor's blood. The blood was then diluted to 6 mL volume using PBS. Post capture, the released cells (FIG. 3C) were then cultured in a 48 well plate (Greiner Bio-One, Monroe, N.C.) in irradiated J2 conditioned F+Y media prepared as described. As shown in FIG. 3D, released cells remained viable at Day 5 and expanded in culture.

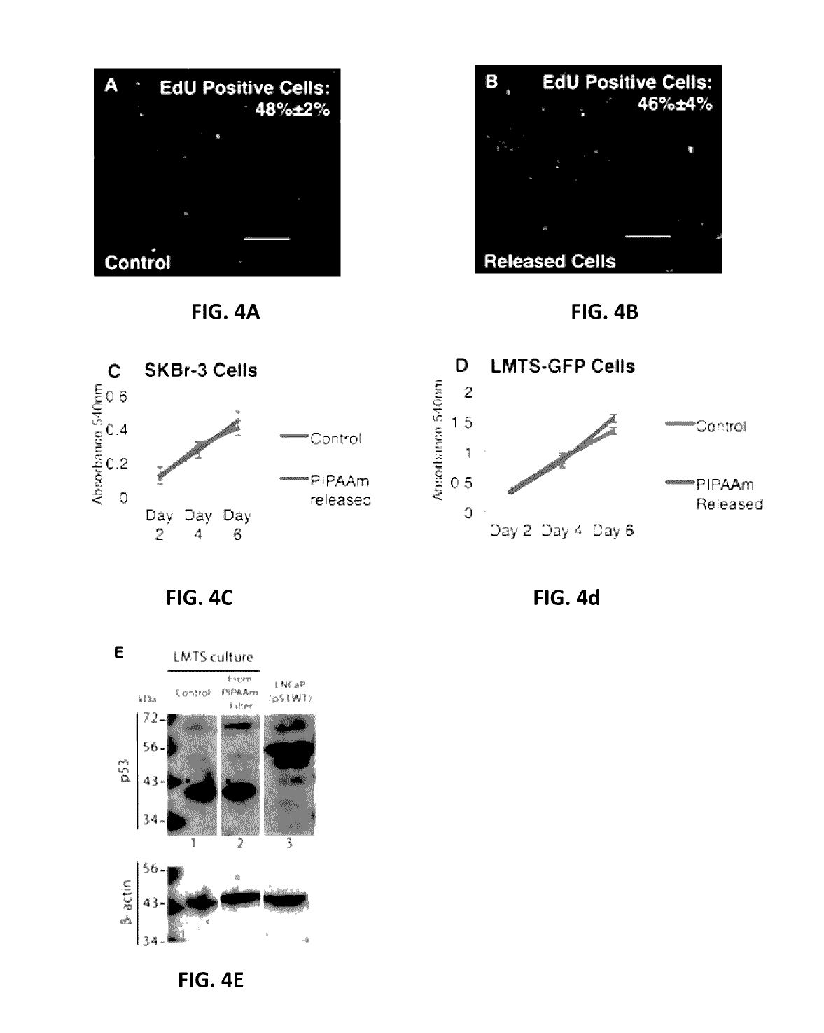

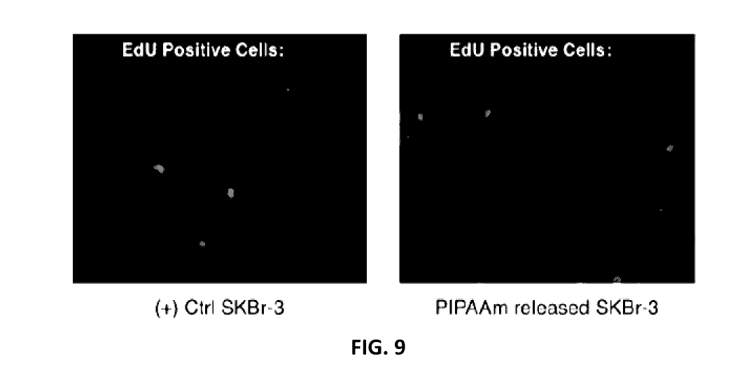

In order to confirm that the process of retrieving tumor cells from blood does not alter the cells' proliferation rate, metabolism and biochemical properties, we analyzed the tumor cells retried from blood by MTT and EdU assays as shown FIG. 4. For the EdU assay, the modified thymidine analog EdU (Life Technologies, Grand Island, N.Y.) was integrated into newly synthesized DNA and then labeled with Alexa Fluor 594® dye. Cells were incubated with EdU for 2 hours and then fixed for labeling. We then enumerated the total number of cells (Hoechst+cells) and newly proliferated cells (Hoeschst+, EdU+cells) and calculated the percentage of EdU+cells. For SKBr-3 cells, EdU+cells constituted 29%±6% of the control cell population and 32%±8% of the PIPAAm released cells population, with no significant difference between groups (p value=0.44) (FIG. 12, Table 4) (FIG. 9). For LMTS-GFP cells, EdU+cells constituted 48%±2% of the control cell population (FIG. 4A) and 46%±4% of the PIPAAm released cells population (FIG. 4B), with no significant difference between groups (p value=0.38). To test the growth curve and metabolism rate of the PIPAAm released cells, we have also performed MTT assay (Life Technologies, Grand Island, N.Y.) on released SKBr-3 cells and LMTS-GFP cells. As seen in FIG. 4C and D, the growth curves and metabolism rates remained the same for both SKBr-3 and LMTS-GFP cells pre- and post-filter capture and release. To verify that the biochemical markers of LMTS-GFP cells were not altered, we probed for a p53 protein that has an E298* truncation mutation harbored by LMTS-GFP cell line in cells we spiked into blood and retrieved using PIPAAm coated slotted filters. As seen in FIG. 4E, LMTS-GFP cells retrieved from blood by PIPAAm coated slotted filters contained the same truncated p53 tumor suppressor protein as seen in control LMTS cells LMTS-GFP cells.

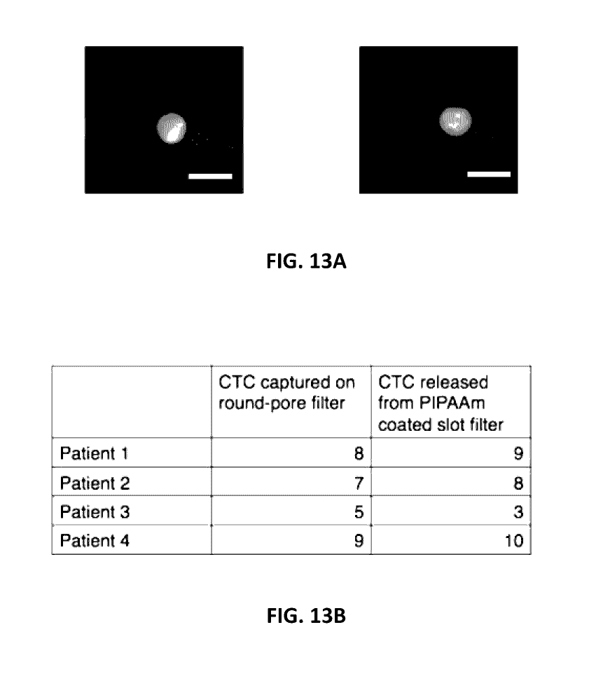

To test the applicability of our strategy for human patient samples, we tested this technology with 4 metastatic breast cancer patient samples. Briefly, we collected 15 mL of peripheral blood by venipuncture from each patient. Blood samples were split evenly into 2 tubes of 7.5 mL blood for each patient. One tube of blood was processed using our round-pore microfilter, which has been demonstrated previously to capture greater number of CTCs as compared with CellSearch® in the same cohort of patients. The other tube was processed using the PIPAAm coated slotted filter. Post-release, the release medium was spun onto a slide for CTC enumeration. Both the microfilter and the slide with released cells were then subjected to immunofluorescence staining for pan-cytokeratin and CD45, markers for CTCs identification. As data shown in FIG. 13B, the CTC enumeration using PIPAAm coated slotted filter or round-pore filter in parallel samples were comparable. Thus, with the PIPAAm coating, we showed the ability to recover CTCs captured from human patient samples with a high retrieval rate.

This detailed description is to be construed as an example only and does not describe every possible embodiment, as describing every possible embodiment would be impractical, if not impossible. One could implement numerous alternate embodiments, using either current technology or technology developed after the filing date of this application.

What is claimed:

1. A system for capturing and releasing cells from a sample, the system comprising:
a microfilter device comprising a membrane filter having a substrate and an array of filtering holes each of a predetermined shape and dimension, the filtering holes forming an array pattern in the substrate, the substrate having a first surface and an opposing second surface, the first surface being positioned to provide a cell capture surface; and
a poly(N-isopropylacrylamide) coating (“PIPAAm coating”) deposited on the first surface and functionalized (i) to remain attractive to cells while cells are captured based on size through a non-specific electrostatic binding, during a hydrophilic state in which the PIPAAm coating is at a temperature at or below a capture temperature and (ii) to release the cells through a phase transition, during a hydrophobic state in which the PIPAAm coating is at a temperature at or above a release temperature, where the release temperature is greater than the capture temperature.

2. The method of claim 1, wherein the microfilter device is formed of a parylene substrate.

3. The method of claim 1, wherein the microfilter device is formed of a polycarbonate substrate, (e.g., parylene, polyimide, polyurethane, hydrophilic photoresist such as su-8) or at metal substrate (e.g., nickel).

4. The method of claim 1, wherein the cells are circulating tumor cells.

5. The method of claim 1, wherein the cells are circulating tumor associated cells.

6. The method of claim 5, wherein the tumor associated cells are cancer associated fibroblasts cells.

7. The method of claim 1, wherein the capture temperature is below 32° C.

8. The method of claim 1, wherein the release temperature is above 32° C.

9. The method of claim 1, further comprising a housing module housing the microfilter with deposited PIPAAm coating.

10. The method of claim 9, further comprising a temperature control module adapted to adjust the temperature of the microfilter between being at or below the capture temperature to being at or above the release temperature.

11. The method of claim 10, wherein the temperature control module is adapted to adjust the temperature of the microfilter while the microfilter is within the housing module.

12. The method of claim 10, wherein the temperature control module is adapted to adjust the temperature of the microfilter while the housing module has been adjusted to expose the microfilter.

13. A method for capturing and releasing cells from a sample, the method comprising:
providing the sample to a microfilter device comprising a membrane filter having a substrate and an array of filtering holes each of a predetermined shape and dimension, the filtering holes forming an array pattern in the substrate, the substrate having a first surface and an opposing second surface, the first surface being positioned to provide a cell capture surface, wherein the member is coated with a poly(N-isopropylacrylamide) coating (“PIPAAm coating”) deposited on the first surface;
with the membrane at or below a first temperature corresponding to a hydrophilic state, capturing cells in the membrane through a non-specific electrostatic binding;
selectively heating the membrane to or above a second temperature corresponding to a hydrophobic state and releasing the captured cells during the hydrophilic state, wherein the second temperature is equal to or greater than the first temperature; and
collecting the released captured cells for analysis.
